(19) United States
(12) Patent Application Publication
(10) Pub. No.: US 2007/0100149 A1
(43) Pub. Date: May 3, 2007

(54) PROCESS FOR PREPARING LETROZOLE

(76) Inventors: Venkata Raghavendra Acharyulu Palle, Hyderabad 500 050, A.P. (IN); Ashok Jerambhai Kalaria, Junagadh, Gujarat (IN); Sandip Ashok Shelke, Ahmednagar, Maharashtra (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/555,951

(22) Filed: Nov. 2, 2006

Related U.S. Application Data
(60) Provisional application No. 60/745,879, filed on Apr. 28, 2006.

(30) Foreign Application Priority Data
Nov. 2, 2005 (IN) 1594/CHE/2005

(51) Int. Cl. C07D 249/08

(52) U.S. Cl. 548/262.2

(57) ABSTRACT

A process for preparing letrozole, and purified letrozole.

### INTRODUCTION TO THE INVENTION

The present invention relates to a process for the preparation of letrozole and intermediates thereof.

Letrozole has the chemical name 4,4′-(1H-1,2,4-triazol-1-ylmethylene)dibenzonitrile and is represented by structural Formula I.

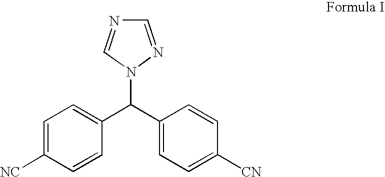

Letrozole is a non-steroidal aromatase inhibitor used for treatment of breast cancer and is commercially available in the market under the brand name FEMARA® in the form of tablets containing 2.5 mg of letrozole.

U.S. Pat. No. 4,978,672 discloses letrozole, its pharmaceutical acceptable salts and compositions containing in letrozole. It also discloses a process for the preparation of letrozole comprising reacting 4-bromomethyl benzonitrile with 1,2,4-triazole to give 4-[1-(1,2,4-triazol-1-yl) methyl benzonitrile; this compound on further reaction with 4-fluorobenzonitrile gives 4-[1-(4-cyanophenyl)-1-(1,2,4-triazol-1-yl)methyl] benzonitrile.

U.S. Pat. No. 5,073,574 discloses the preparation of 4-[α-(4-cyanophenyl)-hydroxy methyl]-benzonitrile, an intermediate useful for the preparation of letrozole. The process comprises the reaction of 4,4′-dibromobenzophenone with CuCN in presence of dimethylformamide to afford 4,4′-dicyanobenophenone. The product is further reacted with sodium borohydride in methanol to give 4-[α-(4-cyanophenyl)-hydroxy methyl]-benzonitrile.

The foregoing processes suffer from serious disadvantages such as low purity of letrozole and use of hazardous or expensive reagents, rendering the processes unsuitable for industrial scale manufacturing.

There is thus a need for a safe and efficient industrial scale process for preparing letrozole that is free of the above-mentioned problems.

According to the present invention there is provided a convenient process for the preparation of letrozole and its intermediates with desired purity and yield by using better preparation techniques, which are simple, ecofriendly, cost-effective, robust and well suited on an industrial scale.

### SUMMARY OF THE INVENTION

The present invention relates to a process for the preparation of letrozole of Formula I.

In one aspect, the present invention relates to a process for the preparation of letrozole of Formula I comprising the steps of:

a) reacting 4,4′-dibromobenzophenone of Formula II with a cyanide reagent to afford 4,4′-dicyanobenzophenone of Formula III;

b) converting 4,4′-dicyanobenzophenone of Formula III into 4,4′-(hydroxymethylene)bis benzonitrile compound of Formula IV using a suitable reducing agent;

c) reacting 4,4′-(hydroxymethylene)bis benzonitrile of Formula IV with p-toluene sulfonyl chloride in the presence of a base to afford toluene-4-sulfonic acid bis-(4-cyano-phenyl)-methyl ester of Formula V; and

d) reacting toluene-4-sulfonic acid bis-(4-cyano-phenyl)-methyl ester of Formula V with 1,2,4-triazole of Formula VI to afford letrozole of Formula I.

In another aspect, the invention relates to a process for the purification letrozole by recrystallizaiton from a suitable solvent.

In another aspect, the present invention provides crystalline letrozole characterized by its X-ray powder diffraction (“XRPD”) pattern, and/or differential scanning calorimetry (“DSC”) curve.

### BRIEF DESCRIPTION OF THE DRAWINGS

FIG. 1 is an X-ray powder diffraction pattern of letrozole prepared according to Example 4.

FIG. 2 is a differential scanning calorimetry curve of letrozole prepared according to Example 4.

FIG. 3 is a thermogravimetric curve analysis curve of letrozole prepared according to Example 4.

### DETAILED DESCRIPTION OF THE INVENTION

The present invention relates to a process for the preparation of letrozole having Formula I.

In one aspect, the present invention relates to a process for the preparation of the letrozole compound of Formula I comprising the steps of:

a) reacting 4,4′-dibromobenzophenone of Formula II with a cyanide reagent to afford 4,4′-dicyanobenzophenone of Formula III;

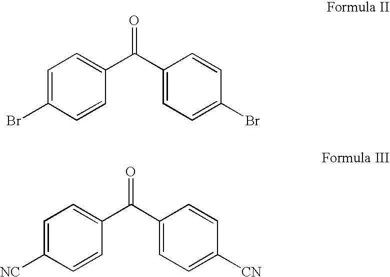

b) converting 4,4′-dicyanobenzophenone of Formula III into 4,4-(hydroxymethylene)bis benzonitrile of Formula IV using a suitable reducing agent;

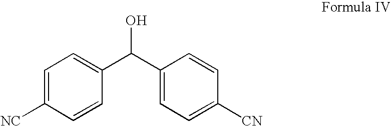

c) reacting 4,4′-(hydroxymethylene)bis benzonitrile of Formula IV with p-toluene sulfonyl chloride in the presence of a base to afford toluene-4-sulfonic acid bis-(4-cyano-phenyl)-methyl ester of Formula V; and

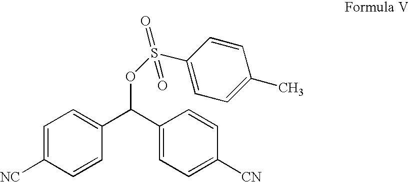

d) reacting toluene-4-sulfonic acid bis-(4-cyano-phenyl)-methyl ester of Formula V with 1,2,4-triazole of Formula VI to afford letrozole of Formula I.

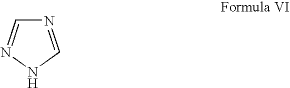

Step a) involves reacting 4,4′-dibromobenzophenone of Formula II with a cyanide reagent to afford 4,4′-dicyanobenzophenone of Formula III.

Suitable cyanide reagents that can be used in the process of step a) include but are not limited to: alkali metal complexes such as potassium ferrocyanide, potassium ferricyanide, and the like; alkali metal cyanides such as sodium cyanide, potassium cyanide, and the like; and metal cyanides such as silver cyanide, and the like.

Suitable bases which can be used in the reaction include but are not limited to metal bicarbonates such as sodium bicarbonate, potassium bicarbonate, and the like. Some of the bases can be used in solution form, prepared either in alcoholic or in aqueous media.

Suitable catalysts which can be used in the above reaction include but are not limited to palladium chloride, nickel, lithium aluminum hydride and the like.

Suitable solvents which can be used in step a) include but are not limited to: ketonic solvents such as acetone, ethyl methyl ketone, methyl isobutyl ketone and the like; esters such as ethyl acetate, n-propyl acetate, n-butyl acetate, t-butyl acetate and the like; nitrile solvents such as acetonitrile, priopionitrile and the like; halogenated solvents such as chloroform, dichloromethane and the like; aprotic solvents such as N,N-dimethylformamide (DMF), dimethylsulfoxide (DMSO), N,N-dimethylacetamide (DMA) and the like; water; and mixtures thereof in various proportions.

Suitable temperatures for conducting the reaction range from about −10 to 150° C., or about 90 to 100° C.

4,4-dicyanobenzophenone of Formula III obtained by the process of the present invention may be isolated and recrystallized in a suitable solvent, if desired.

Suitable solvents which can be used for crystallization or precipitation include but are not limited to: water; hydrocarbons such as n-hexane, cyclohexane, heptane and the like; aromatic solvents including benzene, toluene and the like; alcoholic solvents such as methanol, ethanol, isopropanol, and the like; esters such as ethyl acetate, methyl acetate, isopropyl acetate and the like; and halogenated solvents such as dichloromethane, and the like.

Step b) involves converting 4,4-dicyanobenzophenone of Formula III into 4,4-(hydroxymethylene)bis benzonitrile of Formula IV using a suitable reducing agent.

Suitable reducing agents that can be used in the process of step b) include but are not limited to metal borohydrides such as sodium borohydride, lithium borohydride, and the like;

Suitable solvents which can be used include but are not limited to: alcoholic solvents such as methanol, ethanol, isopropyl alcohol, n-butanol and the like; ketonic solvents such as acetone, ethyl methyl ketone, methyl isobutyl ketone and the like; and esters such as ethyl acetate, n-propyl acetate, n-butyl acetate, t-butyl acetate and the like.

The quantity of reducing agent that is suitable for completion of the reaction ranges from about 0.3 to about 1 moles per mole of 4,4′-dicyanobenzophenone of Formula III.

Suitable temperatures for conducting the reaction range from about −50 to 150° C., or about 0 to 30° C.

After reaction completion, the reaction mixture is neutralized with acid to a pH about 5-8 and extracted with a suitable water immiscible solvent.

Suitable acids for neutralization of above reaction include but are not limited to acetic acid, hydrochloric acid, hydrobromic acid and the like.

4,4′-(hydroxymethylene)bis benzonitrile of Formula IV obtained by the process of the present invention may be isolated by crystallization or precipitation in a suitable solvent.

Suitable solvents which can be used in the crystallization or precipitation include but are not limited to: water; hydrocarbons such as n-hexane, cyclohexane, heptane and the like; aromatic solvents including benzene, toluene and the like; and alcoholic solvents such as methanol, ethanol, isopropyl alcohol, n-butanol and the like.

Step c) involves reacting 4,4′-(hydroxymethylene)bis benzonitrile of Formula IV with p-toluenesulfonyl chloride in the presence of a base to afford toluene-4-sulfonic acid bis-(4-cyano-phenyl)-methyl ester of Formula V.

A suitable quantity of p-toluenesulfonyl chloride that can be used in the process of step c) may range from about 0.5 to about 2 moles per mole of 4,4′-(hydroxymethylene)bis benzonitrile of Formula IV.

Suitable bases that can be used include but are not limited to alkali metal hydroxides, carbonates and bicarbonates such as sodium carbonate, sodium hydroxide, potassium carbonate, potassium hydroxide, sodium bicarbonate, and the like.

Suitable solvents which can be used for the above reaction include but are not limited to: water; ketonic solvents such as acetone, ethyl methyl ketone, methyl isobutyl ketone and the like; and esters such as ethyl acetate, n-propyl acetate, n-butyl acetate, t-butyl acetate and the like.

Step d) involves reacting toluene-4-sulfonic acid bis-(4-cyano-phenyl)-methyl ester of Formula V with 1,2,4-triazole of Formula VI to afford letrozole of Formula I.

The quantity of 1,2,4-triazole of Formula VI that can be used for the preparation of Formula I ranges from about 2 to about 6 moles per mole of toluene-4-sulfonic acid bis-(4-cyano-phenyl)-methyl ester of Formula V.

Phase transfer catalyst (PTC) methodology, which can be used in the preparation of letrozole, involves a substrate (which is soluble in an organic layer) and an anionic reagent (often a nucleophile), which is dissolved in an aqueous layer. The substrate and the anion are then brought together by a catalyst, which transports the anion into the organic phase where reaction can take place with the substrate.

Quaternary ammonium and phosphonium salts, with their unique capability to dissolve in both aqueous and organic liquids, are the catalysts of choice for many phase transfer applications. The ammonium based phase transfer catalysts include tetrabutylammonium bromide, tetrabutylammonium chloride, tetrabutylammonium fluoride trihydrate, tetrabutylammonium fluoride solution, tetrabutylammonium hydrogen sulfate, tetrabutylammonium iodide, tetrabutylammonium thiocyanate, tetrabutylammonium tetrafluoroborate, benzyltributylammonium chloride, benzyltriethylammonium chloride, benzyltrimethylammonium bromide, benzyltrimethylammonium chloride, hexadecyltrimethylammonium bromide, hexadecyltrimethylammonium chloride, hexadecyltrimethylammonium chloride solution, hexadecyltrimethylammonium hydrogen sulfate, methyltrioctadecylammonium bromide, methyltrioctylammonium bromide, methyltrioctylammonium chloride, tetraethylammonium bromide, tetraethylammonium chloride, tetraethylammonium fluoride dihydrate, tetraethylammonium hexafluorophosphate, tetraethylammonium tetrafluoroborate, tetrahexylammonium hydrogen sulfate, tetramethylammonium bromide, tetramethylammonium chloride, tetraoctylammonium bromide, tetraoctylammonium chloride and the like. Phosphonium based phase transfer catalysts include tributylhexadecylphosphonium bromide, tetrabutylphosphonium chloride, tetrabutylphosphonium bromide and the like. Also useful are crown ethers and polyethylene glycols such as 12-crown-4, 1-aza-15-crown-5, 15-crown-5, and the like.

PTCs are used in step d) to increase reaction rate, to permit lower reaction temperatures, to avoid the need for expensive anhydrous or aprotic solvents and to permit use of water together with an organic solvent as a reaction medium. The quantity of PTC that is used in the above reaction is to about 0.5 to about 1 moles per mole of toluene-4-sulfonic acid bis-(4-cyano-phenyl)-methyl ester of Formula V.

Suitable bases, which can be used for providing letrozole, include but are not limited to alkali metal hydroxides, carbonates and bicarbonates like sodium carbonate, sodium hydroxide, potassium carbonate, potassium hydroxide, sodium bicarbonate, and the like. Due to the presence of a base, the triazole can form a salt.

Suitable solvents which can be used in the process of step d) include but are not limited to ketonic solvents such as ethyl methyl ketone, methyl isobutyl ketone and the like; and esters such as ethyl acetate, n-propyl acetate, n-butyl acetate, t-butyl acetate and the like; chlorinated solvents such as dichloromethane and the like.

After reaction completion, the reaction mixture is separated into two layers and the aqueous layer is extracted with a suitable water immiscible solvent.

Suitable solvents which can be used for extraction of letrozole include but are not limited to: ketonic solvents such as acetone, methyl isobutyl ketone, ethyl methyl ketone, and the like; chlorinated solvents such as dichloromethane, dichloroethane, chloroform, and the like; esters such as ethyl acetate, methyl acetate and the like; ethers such as diethyl ether, diisopropyl ether, and the like; and hydrocarbons such as n-hexane, cyclohexane, heptane and the like.

The organic layer comprising letrozole is treated with a suitable inorganic acid to extract the product into the aqueous layer.

Suitable acids which can be used for extraction include but are not limited to inorganic acids such as hydrochloric acid, hydrobromic acid and the like.

The aqueous layer comprising letrozole is treated with a suitable inorganic base and the product is extracted with a suitable solvent.

Suitable bases that can be used include but are not limited to sodium or potassium hydroxide, bicarbonate or carbonate, ammonia, and the like.

Suitable solvents which can be used for extraction of letrozole include but are not limited to chlorinated solvents such as dichloromethane, dichloroethane, chloroform, and the like;

The organic layer comprising letrozole may be used directly in the next processing step, or it can be concentrated to form a residue, or letrozole can be isolated by precipitation using a suitable solvent such as water.

Letrozole obtained at this stage may contain some isomeric and structure related impurities such as the compounds having Formula VII, Formula VII, Formula IX and Formula X. In an embodiment of the present invention, there is also provided a process for the purification of letrozole to reducing concentrations of the isomeric impurities and structural related impurities to ≦0.1%, which process comprises:

a) providing a solution of crude letrozole in a suitable solvent;

b) passing the solution of step a) through a silica gel bed; and

c) recovering purified letrozole.

The step of providing a solution of crude letrozole includes dissolving solid letrozole in a suitable solvent or obtaining a solution comprising crude letrozole from a previous processing step such as from synthesis of letrozole.

Suitable solvents which can be used for providing a solution of crude letrozole include but are not limited to: chlorinated solvents such as dichloromethane, dichloroethane, chloroform, and the like; esters such as ethyl acetate, methyl acetate and the like; ethers such as diethyl ether, diisopropyl ether, and the like; and hydrocarbons such as n-hexane, cyclohexane, heptane and the like.

Step b) involves passing the solution obtained from step a) through a silica gel bed.

Silica gel that is used to prepare a bed can have a particle size range about 240-400 mesh, 100-200 mesh, or 60-120 mesh. The quantity of silica gel used can range from about 4 to 15 times, or about 4 to 6 times, the weight of the starting compound of Formula II. Larger amounts of silica gel can also be used.

Suitably, synthetic resins may be used instead of silica gel to remove impurities in the said process. Synthetic resins include divinylbenzene-styrene copolymers, copolymers of divinylbenzene, styrene and other derivatives of these having aliphatic and/or aromatic moieties comprising from 2 to 18 carbon atoms, or having substituted halogen atoms chlorine, fluorine or bromine, copolymers of divinylbenzene and styrene with surface grafted moieties that are aliphatic or aromatic containing two or more carbon atoms and/or having substituted halogen atoms chlorine, fluorine or bromine, resins based on one or a combination of natural polymers, derivatized or not, for example, agarose, dextran or cellulose, and resins based on a polymethacrylate matrix or its combination with other acrylate polymers, prepared by cross-linking of monomers, with or without grafted moieties that are aliphatic or aromatic containing two or more carbon atoms with or without substituted halogen atoms chlorine, fluorine or bromine.

Step (c) involves recovering purified letrozole.

In an embodiment, purified letrozole is recovered by concentrating the filtrate obtained from the previous step using suitable techniques.

Suitable techniques which can be used for the concentration include distillation using a rotational evaporator device such as a Buchi Rotavapor, spray drying, agitated thin film drying (“ATFD”), and the like.

Distillation of the solvent may be conducted under a vacuum, such as below about 100 mm Hg to below about 600 mm Hg, at elevated temperatures such as about 20° C. to about 70° C. Any temperature and vacuum conditions can be used as long as there is no increase in the impurity levels of the product.

In another aspect, the present invention relates to a process for the purification of letrozole comprising crystallizing from a mixture comprising an alcohol and water.

The process comprises the steps of:

1) providing a solution of letrozole in a solvent comprising an alcohol;

2) combining water with the solution of step 1); and

3) recovering crystalline letrozole.

The step of providing a solution of letrozole includes dissolving letrozole in an alcohol or obtaining the solution comprising letrozole from a previous processing step.

Any form of letrozole is acceptable for providing a solution, such as any crystalline or amorphous form of letrozole.

The concentration of letrozole in the alcohol solution is not critical as long as sufficient alcohol is employed to ensure total dissolution. The amount of solvent employed is usually kept to a minimum so as to avoid excessive product loss during crystallization and isolation.

Alcohols that can be used in the above dissolution step include but are not limited to methanol, ethanol, isopropyl alcohol, n-butanol and the like.

The solution can be prepared at temperatures ranging from about 25° C. to 100° C. Depending on the quantity of solvent taken, solute may dissolve at 25 to 100° C., or the solution may need to be heated to elevated temperatures of about 50° C. to 100° C.

The solution can be optionally treated with activated charcoal to enhance the color of the compound and filtered through a medium such as a flux calcined diatomaceous earth (“Hyflow”) bed to remove the carbon.

The solution can optionally be filtered by passing through paper, glass fiber, or other membrane material or a clarifying agent such as celite. Depending upon the equipment used and the concentration and temperature of the solution, the filtration apparatus may need to be heated to avoid premature crystallization.

Step 2) involves combining water with the solution of step 1).

The quantity of water that can be used in the process of step 2) will be at least about 3 times the weight of letrozole.

The reaction mass may be maintained further at temperatures to about 0° C. to below 45° C., for a period of time as required for a more complete isolation of the product. The exact cooling temperature and time required for complete crystallization can be readily determined by a person skilled in the art and will also depend on parameters such as concentration and temperature of the solution or slurry.

In alternative embodiments, the water can be added to the solution of step 1), or the solution of step 1) can be added to the water.

Step 3) involves recovering crystalline letrozole.

The solid can be isolated by conventional techniques such as filtering, decanting, centrifuging and the like, or by filtering under an inert atmosphere using gases such as for example nitrogen and the like.

The wet cake obtained may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods until a desired product purity is obtained. Useful drying times frequently are from about 1 to 20 hours, or longer.

Letrozole obtained by the above process was analyzed by using high performance liquid chromatography (“HPLC”) with the conditions described Table 1. The method is described in United States Pharmaceopeia 29, United States Pharmacopeial Convention, Inc., Rockville, Md., 2005, pages 1232-1234.

TABLE 1

| Column and | Intertsil ODS 3V, 150 × 4.6 mm, 5 μm | | | |
|---|---|---|---|---|
| Packing: | | | | |
| Mobile Phase | Filtered and degassed Milli Q water. | | | |
| A: | | | | |
| Mobile Phase | Filtered and degassed acetonitrile. | | | |
| B: | | | | |
| | | Solution | Solution | |
| | Time | A (% | B (% | |
| | (minutes) | v/v) | v/v) | Elution |
| Gradient: | 0 | 70 | 30 | Isocratic |
| | 25 | 50 | 50 | Linear |
| | | | | gradient |
| | 35 | 30 | 70 | Linear |
| | | | | gradient |
| | 50 | 30 | 70 | Isocratic |
| | 51 | 70 | 30 | Equilibration |
| | 60 | 70 | 30 | Re- |
| | | | | equilibration |

| | |
|---|---|
| Flow rate: | 1.0 ml/minute |
| Wavelength of | 230 & 265 nm |
| detection: | |
| Temperature: | 35 ± 2° C. |
| Injection | 15 μL |
| volume: | |
| Diluent: | Acetonitrile |
| Run time: | 60 minutes |

The relative retention times (RRT) of impurity peaks are given below, where let letrozole is assigned the value of 1.

| Impurity | RRT |
|---|---|
| Letrozole related compound A | 0.58 |
| 4,4′,4″- | 2.69 |
| methylidenetrisbenzonitrile | |
| 4,4′-dicyanobenzophenone | 1.17 |
| 4,4′-(hydroxy methylene) bis | 1.13 |
| benzonitrile | |
| Methane sulfonic acid bis-(4- | 2.87 |
| cyano-phenyl)-methyl ester | |
| 4,4′-methlenebisbenzonitrile | 2.11 |
| 4-[(4-Bromo-phenyl)-[1,2,4] | 1.85 |
| triazol-1-yl-methyl]-benzonitrile | |
| 4,4′-dibromobenzophenone | 3.45 |
| Letrozole | 1.00 |

Letrozole obtained by the process of present invention has low levels of any one or more of the following structural related impurities:

1) 4-4′-(4H-1,2,4-Triazol-4-ylmethylene)dibenzonitrile of Formula VII (“letrozole related compound A”);

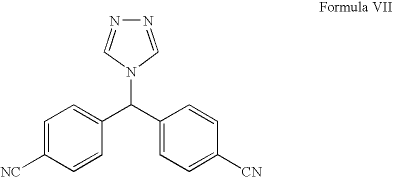

2) 4-[(4-Bromo-phenyl)-[1,2,4]triazol-1-yl-methyl]-benzonitrile of Formula VIII;

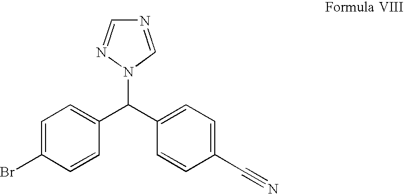

3) 4,4′,4″-methyllidenetrisbenzonitrile of Formula IX;

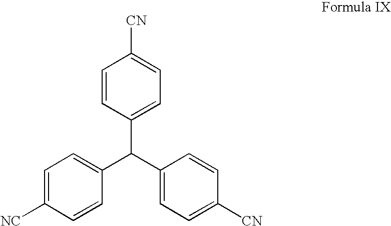

4) 4,4′-dicyanobenzophenone of Formula III;

5) 4,4′-(hydroxy methylene)bis benzonitrile of Formula IV; and

6) Methane sulfonic acid bis-(4-cyano-phenyl)-methyl ester of Formula X.

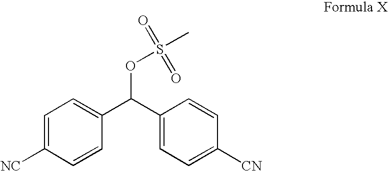

Yet another aspect of the present invention provides crystalline letrozole characterized by its X-ray powder diffraction (“XRPD”) pattern, and/or differential scanning calorimetry (“DSC”) curve.

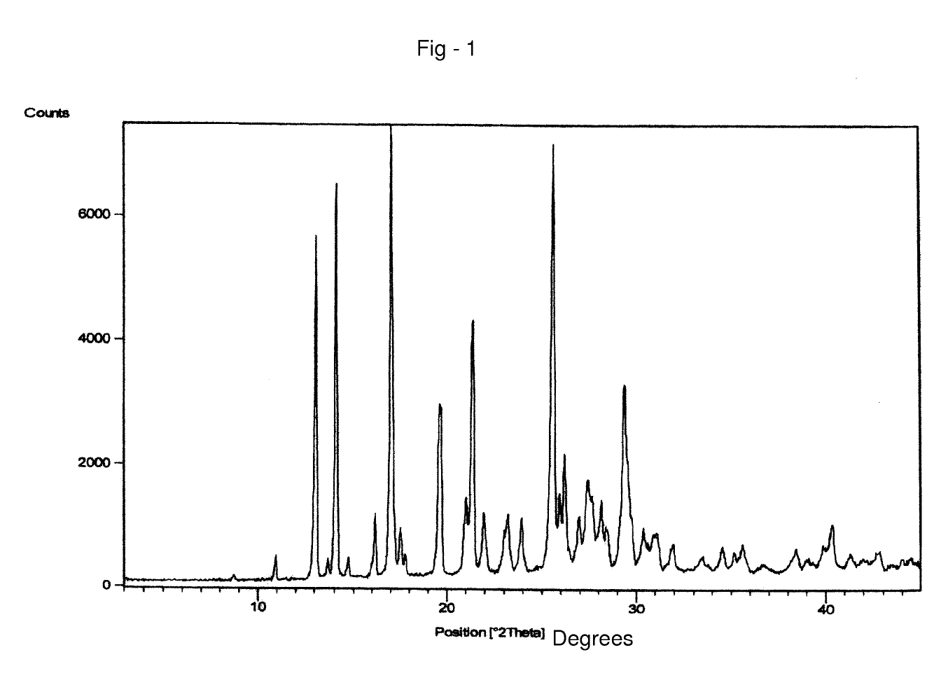

Crystalline letrozole obtained in the present invention is characterized by its XRPD pattern substantially in accordance with the pattern of FIG. 1. All XRPD data reported herein were obtained using Cu Ka radiation, having the wavelength 1.541 Å and were obtained using a Bruker Axe D8 Advance Powder X-ray Diffractometer.

Crystalline letrozole is characterized by an XRPD diffraction pattern comprising characteristic peaks at about 13.1, 14.1, 17.1, 19.6, 21.4, 25.6, 26.2, 27.4, and 29.3, ±0.2 degrees two theta.

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C.

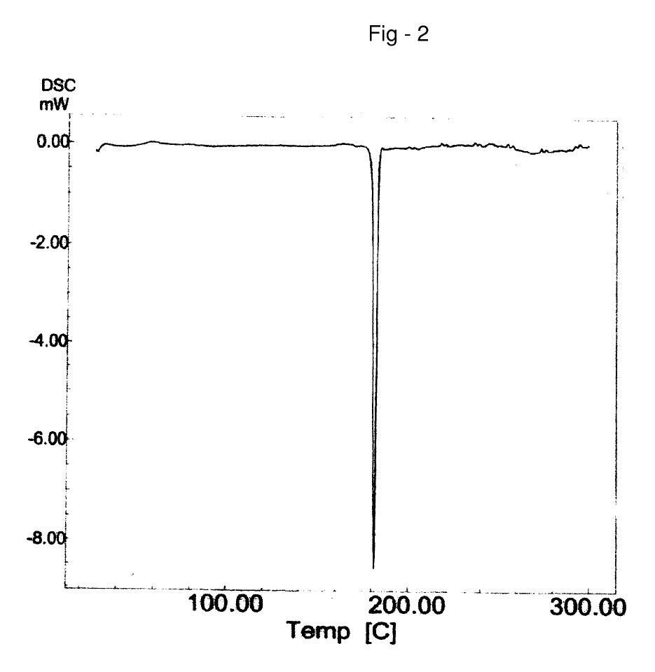

Crystalline letrozole has a characteristic differential scanning calorimetry curve substantially in accordance with FIG. 2, having an endothermic peak at about 181° C. (onset about 180° C. and endset about 183° C.).

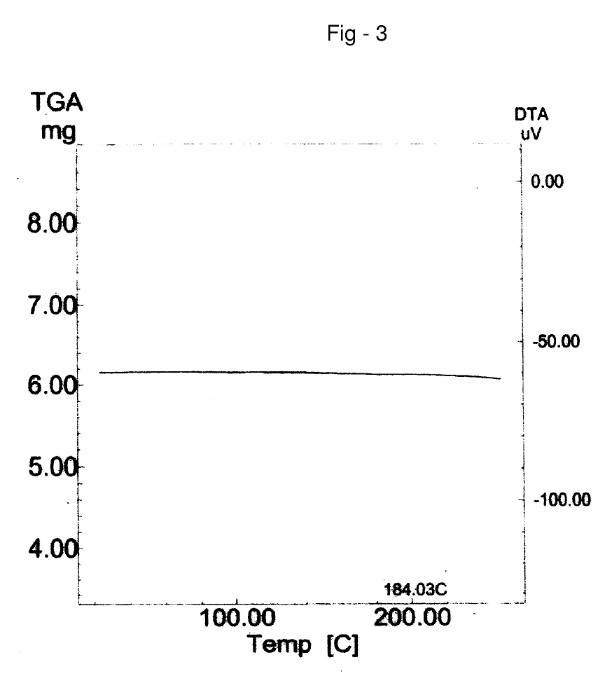

Crystalline letrozole has a characteristic thermogravimetric curve (TGA) substantially in accordance with FIG. 3, having an endothermic peak of about −0.15%.

In another embodiment letrozole obtained by the process of present invention has a particle size D₉₀ less than about 500 microns, or about 300 microns, or about 150 microns.

The D₁₀, D₅₀ and D₉₀ values are useful ways for indicating a particle size distribution. D_{π} refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀ and D₉₀ include laser diffraction, such as using laser light scattering equipment from Malvern Instruments Ltd. of Malvern, Worcestershire, United Kingdom. There is no specific lower limit for any of the D values.

In yet another aspect the invention provides letrozole substantially free of residual solvents.

Letrozole obtained using the process of the present invention has a residual solvent content that is within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

Letrozole obtained in this invention contains less than about 100 ppm or less than about 500 ppm of methanol, less than about 100 ppm or less than about 500 ppm of n-hexane, less than about 100 ppm or less than about 500 ppm of dichloromethane, less than about 100 ppm or less than about 500 ppm of acetone, less than about 100 ppm or less than about 500 ppm of N,N-dimethylacetamide, less than about 100 ppm or less than about 500 ppm of methyl isobutyl ketone, and less than about 100 ppm or less than about 500 ppm of N,N-dimethyl formamide.

Certain specific aspects and embodiments of the present invention will be explained in more detail with reference to the following examples, which are provided by way of illustration only and should not be construed as limiting the scope of the invention in any manner.

### EXAMPLE 1

### PREPARATION OF 4,4′-DICYANOBENZOPHENONE OF FORMULA III

100 g of 4,4-dibromobenzophenone were suspended in 1000 ml of N,N-dimethylacetamide (DMA) and stirred for 10 minutes at 25-35° C. 93.2 g of potassium ferrocyanide, 62.5 g of sodium carbonate and 512 mg of palladium chloride were charged to the suspension. The suspension was heated to 95-100° C. and stirred for 6 hours at 95-100° C. Then the reaction mass was cooled to 27° C. and filtered through a celite bed followed by washing the celite bed with 100 ml of N,N-dimethylacetamide (DMA). 2 L of methanol were charged to the filtrate and stirred for 2 hours at 25° C., followed by filtering at 25° C. Methanol was distilled from the filtrate and 1.1 L of water was charged at 25° C. The resultant suspension was filtered followed by washing the solid with 100 ml of water and drying the solid at 58° C. under vacuum for 4 hours, to afford 54 g of title compound with a purity by HPLC of 98.99% and a water content by the Karl Fischer method of 0.08%.

### EXAMPLE 2

### PREPARATION OF 4,4′-(HYDROXY METHYLENE)BISBENZONITRILE OF FORMULA IV

200 ml of methanol was taken into a round bottom flask followed by charging 40 g of 4,4-dicyanobenzophenone and stirring for about 10 minutes. 3.28 g of sodium borohydride was added slowly to the above suspension at 0-5° C. followed by stirring at 25-30° C. for 30 minutes, and the formed solution was neutralized with 25 ml of glacial acetic acid to a pH of 6.33. 800 ml of water was charged to the above-neutralized suspension and stirred for 30 minutes, and the precipitate was filtered, washed with 400 ml of water and finally subjected to drying at 55-60° C. under vacuum for 4 hours to afford 40 g of title compound having a purity by HPLC of 99.36% and a water content by the Karl Fischer method of 0.27%.

### EXAMPLE 3

### PREPARATION OF TOLUENE SULPHONIC ACID BIS-(4-CYANOPHENYL)METHYL ESTER OF FORMULA V

250 ml of acetone was taken into a round bottom flask to which was then added 50 g of 4,4-(hydroxymethylene)bis benzonitrile and the mixture was stirred for 10 minutes. The obtained solution was cooled to 0-5° C. and stirred for 5 minutes. 53.5 g of p-toluenesulphonyl chloride dissolved in 100 ml of acetone was slowly added to the above solution and stirred for 5 minutes followed by the dropwise addition of 200 ml of 2N aqueous sodium hydroxide at 0-5° C. over 45 minutes. The reaction mass was subjected to stirring for 30 minutes at 0 to 5° C. The obtained solid mass was filtered, washed with 100 ml of water and suction dried for 1 hour to afford 78 g of title compound having purity by HPLC of 97.36% and a water content by the Karl Fischer method of 1.07% w/w.

### EXAMPLE 4

### PREPARATION OF LETROZOLE OF FORMULA I

700 ml of water was taken into a round bottom flask to which was then added 49.8 g of 1,2,4-triazole with stirring for about 5 minutes, and then 99.6 g of potassium carbonate was added with stirring for 10 minutes. 350 ml of methyl isobutyl ketone (MIBK) and 58.1 g of tetrabutylammonium bromide were charged and stirred for 2 hours. 70 g of toluene sulphonic acid bis-(4-cyano phenyl)methyl ester was charged to the obtained reaction mixture at 20-30° C. and stirred for 48 hours. The formed aqueous and organic layers were separated and the aqueous layer extracted with 140 ml of methyl isobutyl ketone (MIBK) followed by washing the obtained organic layer with 700 ml of water. 1050 ml of 5N hydrochloric acid was charged to the organic layer, and stirred for 60 minutes. The formed aqueous and organic layers were separated and the organic layer extracted with 140 ml of aqueous 5N hydrochloric acid followed by washing the aqueous layer with 2×40 ml of n-hexane. The aqueous layer was basified with 350 g of sodium carbonate to get a pH of about 8.5 at a temperature below 25° C. followed by extraction with 2×350 ml of dichloromethane and the organic layer was washed with 700 ml of water. The resultant organic layer was distilled completely at below 40° C. and 350 ml of methanol and 1050 ml of water were charged, and stirred for 1 hour at 10-15° C. The formed precipitate was filtered followed by washing the solid with 350 ml of water, and suction drying of the solid for 30 minutes to afford 97.42% pure title compound.

Dissolved the above-obtained letrozole compound in 140 ml of dichloromethane. A silica gel bed was prepared, using 140 g of silica gel (230-400 mesh) on a fritted glass filter funnel (10 cm height and 10 cm diameter), and the silica bed was washed with 1.4 L of dichloromethane. The dichloromethane solution was passed through the silica gel bed and the bed washed with 28 L of dichloromethane. The obtained filtrate was concentrated below 40° C. to a volume of about 140 ml of dichloromethane then 500 ml of methanol was charged and stirred for 10 minutes. The obtained solution was concentrated below 40° C. to a volume of 105-120 ml and 350 ml of water was charged and stirred for 1 hour. The formed solid was filtered followed by washing the solid with 140 ml of water and drying the solid at 55-60° C. for 4 hours under reduced pressure to afford 11.4 g of title compound having purity by HPLC of 99.8% and all other impurities 0.15%.

Impurities:

Letrozole related compound A of Formula VII: not detected.

4,4′,4″-methylidenetrisbenzonitrile of Formula IX: not detected.

4,4′-dicyanobenzophenone of Formula III: not detected.

4,4′-(hydroxy methylene)bis benzonitrile of Formula IV: not detected.

Methane sulfonic acid bis-(4-cyano-phenyl)-methyl ester of Formula X: not detected.

Highest unidentified impurity: 0.05%

We claim:

1. A process for preparing letrozole, comprising reacting 4,4-(hydroxymethylene)bis benzonitrile with p-toluenesulfonyl chloride to form an ester.

2. The process of claim 1, wherein an ester has a formula:

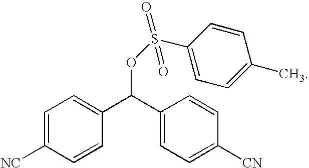

3. The process of claim 1, wherein 4,4-(hydroxymethylene)bis benzonitrile is prepared by reacting 4,4′-dibromobenzophenone with a cyanide reagent to form an intermediate, and reducing the intermediate.

4. The process of claim 3, wherein a cyanide reagent comprises potassium ferrocyanide, potassium ferricyanide, sodium cyanide, potassium cyanide, or silver cyanide.

5. The process of claim 3, wherein reducing comprises reacting with a metal borohydride.

6. The process of claim 1, further comprising reacting an ester with 1,2,4-triazole to form letrozole.

7. The process of claim 6, wherein reacting an ester occurs in the presence of a phase transfer catalyst.

8. The process of claim 6, wherein reacting an ester occurs in the presence of tetrabutylammonium bromide.

9. A process for purifying letrozole, comprising crystallizing letrozole from a solution comprising an alcohol by combining a solution with water.

10. The process of claim 9, wherein an alcohol comprises methanol, ethanol, isopropyl alcohol, or n-butanol.

11. The process of claim 9, wherein an alcohol comprises methanol.

12. The process of claim 9, wherein the amount of water is at least about 3 times a weight of letrozole in a solution.

13. The process of claim 9, wherein combining comprises adding water to a solution.

14. A process for purifying letrozole. comprising passing a solution comprising letrozole through silica gel.

15. The process of claim 14, wherein a solution comprises a chlorinated hydrocarbon as a solvent.

16. The process of claim 14, wherein a solution comprises a dichloromethane as a solvent.

17. A compound having a formula: [chemical structure]
